(19) United States
(12) Patent Application Publication
(10) Pub. No.: US 2009/0270472 A1
(43) Pub. Date: Oct. 29, 2009

(54) METHOD TO USE OXYMETAZOLINE TO INTERRUPT THE DEVELOPMENT OF THE COMMON COLD SYNDROME

(76) Inventor: KENNETH GOULD, Los Angeles, CA (US)

Correspondence Address: Kenneth Gould M.D., 3314 Club Drive, Los Angeles, CA, 90064, US

(21) Appl. No.: 12/404,428

(22) Filed: Mar. 16, 2009

Related U.S. Application Data
(60) Provisional application No. 61/125,392, filed on Apr. 25, 2008.

(51) Int. Cl. A61K 31/4164; A61P 27/14

(52) U.S. Cl. 514/385

(57) ABSTRACT

This invention describes a new manner of treating the common cold. It claims that treatment with Oxymetazoline nasal spray at the time of onset of first cold symptom(s) can interrupt the development of the common cold syndrome and result in cure of the cold.

### CROSS REFERENCE TO PROVISIONAL APPLICATION

No. 61/125,392

### CROSS-REFERENCE TO RELATED APPLICATIONS

Not applicable

### STATEMENT REGARDING FEDERALLY SPONSORED RESEARCH OF DEVELOPMENT

Not applicable

### REFERENCE TO SEQUENCE LISTING

Not applicable

### BACKGROUND OF THE INVENTION

Treatment of the common cold is generally regarded as treatment for the symptoms associated with this infection. There are claims for interventions which are said to block the development or to significantly diminish the duration of symptoms of the cold syndrome. Some of these have proven too expensive or toxic for use while others have shown inconsistent results when studies were repeated and scrutinized with scientific rigor. There is no generally accepted cure for the common cold

The author of this patent, an Infectious Diseases Specialist, has found that one of the agents commonly used for treatment of the nasal congestion, rhinitis and rhinorrhea associated with the common cold can interrupt the development of the cold syndrome when it is administered as a nasal spray within one (1) hour after the recognition of the first cold symptom(s).

The basis of this invention is the use of Oxymetazoline 0.05% spray at a time in cold onset which is earlier than commonly recommended in order to achieve an effect that has not been previously thought possible. Presented here is a novel approach to the treatment of the common cold which seeks to interrupt or cure the infection with an agent that has been licensed and commonly used only for the amelioration or control of nasal symptoms.

### FIELD OF THE INVENTION

The invention relates to the treatment of the common cold. More specifically it relates to an early intervention with a nasal spray comprising an alpha adrenergic agent, Oxymetazoline. This and multiple similar agents are often used to treat the nasal congestion and runny nose associated with the common cold. This medication is the active agent in many brand name (Afrin, Dristan, Vicks) nasal sprays as well as in many generic brands. They are all sold without prescription as over the counter decongestant products.

Observation and therapeutic intervention has demonstrated that early use of this agent, at a time prior to the usual and customary time of use, can cure/arrest/abort the development of the full syndrome. If further study confirms this observation and if there is widespread acceptance and use of this invention, significant societal benefit would be derived.

### DESCRIPTION OF THE ART

The “common cold” is a term used to describe a respiratory illness caused by infection of a human with a respiratory virus.

Since multiple families of virus may be associated with this infection specific therapies to target a given virus or to immunize against these infections have been difficult to develop.

The common cold is the most frequent infection in humans and is estimated to cause 500 million illnesses yearly in the United States. This burden of illness leads to 100 million Physician visits, $2.9 billion spent on over the counter medications, and $400 million spent on prescription medications each year. There are 189 million school days missed by children suffering from colds, and their parents miss 126 million work days while caring for them. In addition, millions of employees also miss days due to the common cold. In the United States, it is estimated that the total economic cost of this infection exceeds $20 billion per year.

The term “common cold syndrome” refers to a complex of symptoms. This may include some or all of the following: sore throat, nasal congestion, chest congestion, runny nose, increased mucus production, sneezing, cough, fever, chills, headache, weakness, and tiredness. The syndrome may start with any of the above and may progress with development of many or all of the above symptoms. On average the syndrome last for 7 days. Complications of the cold can lead to more serious problems such as sinusitis, ear infection, and pneumonia.

The common cold is an infection that begins after contact/contamination of the individual with a virus that has the ability to infect the respiratory mucosa. There are multiple types of virus capable of causing this syndrome. They include the rhinovirus, coronavirus, adenovirus and others. 40-50% of colds are due to the rhinovirus of which there are more than 100 serotypes. The virus may be inhaled in droplet form or may reach the nasal or oral mucosa via direct contact with contaminated hands, utensils or other objects. When the virus reaches the target mucosa a cascade of inflammatory responses is initiated by the body in response to the invading virus.

Many of the symptoms and signs of the common cold result from this response. An agent that is used in an attempt to cure/interrupt the development of the cold syndrome must act early enough to block initiation of the multiple inflammatory cascades. Once these cascades are initiated, therapy commonly focuses on amelioration of symptoms through the use of multiple medications, either alone or in combination. These medications include antipyretics, anti-inflammatory agents, antihistamines, anticholinergics, decongestants, anti-tussives, expectorants, and mucolytics.

An “alpha adrenergic agent” refers to a class of compounds that selectively binds to and activates the adrenergic receptors in various tissues within the human body. These agents may also be referred to as alpha adrenergic agonists to differentiate them from alpha adrenergic antagonists that have an opposite effect on the receptors. The activation of these receptors leads to vascular constriction and the contraction of smooth muscles which together lead to decongestion.

Oxymetazoline is an example of an alpha adrenergic agent. Other examples include phenylephrine, xylometazoline, naphazoline, 1-desoxyephedrine, ephedrine, propylhexedrine, pseudoephedrine, and phenylpropanolamine.

There have been many attempts to develop agents to cure the common cold. Many different approaches and agents have been studied. Some of these include the use of:

an interferon, Douglas et al., Prophylactic Efficacy of Intranasal Alpha-Interferon Against Rhinovirus Infection in the Family Setting, The New England Journal of Medicine, 314, pp. 65-70, 1986;

a bradykinin antagonist, Higgins et al., A Study of the Efficacy of the Bradykinin Antagonist, NPC567; in Rhinovirus Infections in Human Volunteers, Antiviral Research vol. 14, pp. 339-344, 1990;

a glucocorticoid, Parr et al., A Randomized Controlled Trial of Glucocorticoid Prophylaxis Against Experimental Rhinovirus Infection, Journal of Infectious Diseases, vol. 162, pp. 1173-1177, 1990;

nedocromil, Barrow et al., The Effect of Intranasal Nedocromil Sodium on Viral Upper Respiratory Tract Infections in Human Volunteers, Clinical and Experimental Allergy, vol. 20, pp. 45-51, 1990;

a combination of interferon-.alpha.sub.2, ipratropium and naproxen, Gwaltney, Combined Antiviral and Antimmediator Treatment of Rhinovirus Colds, The Journal of Infectious Diseases vol. 166, pp. 776-782, 1992;

A zinc salt, Potter et al., DIAS Rounds, Zinc Lozenges for Treatment of Common Colds, The Annals of Pharmacotherapy, vol. 27, pp. 589-592, 1993.

There have been patents awarded for some of these as well as for other inventions which attempt to cure/interrupt the cold syndrome. These include agents which attack the virus by killing, inhibiting, or entrapping it.

A combination of an antiviral agent and antimmediators was described (U.S. Pat. Nos. 5,240,694; 5,422,097; 5,492,689) by Gwaltney in which Interferon alpha-2 was combined with Ipratropium and Naproxyn in an agent called “Covam”. When used in human subjects this agent decreased the incidence and severity of colds after viral challenge. In a second phase of study an alpha adrenergic agent and an antihistamine were added to the “Covam” therapy. Those treated with the new combination did not experience a further decrease in colds but did have a marked reduction in subjective complaints of rhinorrhea and amount of nasal secretions. Investigators did not observe a decrease in progression to a cold syndrome with the addition of the alpha adrenergic agent (phenylephrine HCL), and there was no study performed in which an alpha adrenergic was used a single agent. “Covam” was never developed as a commercial agent because of cost and toxicity considerations.

Other methods for cure of the common cold have included salts or ionic preparations including Zinc, and other metals. In one of these, Eby (U.S. Pat. No. 4,503,070) described the use of zinc gluconate as a “Cure for Common Cold”. This patent does not mention the use of an alpha adrenergic agent alone or in combination with their agent. Various zinc compounds administered via lozenge, gel, and spray formulation have been tested and some have been marketed as medications to decrease the severity of the common cold. Scientific publications have raised concerns about possible toxicity as well as the efficacy of these products (Zicam). U.S. Pat. No. 5,622,724 by Bryce-Smith presents unchelated zinc ion in spray form “to provide a safe, cheap and efficient cure for the common cold”.

U.S. Pat. No. 4,689,223 has been granted for the use of a solution of Sodium bisulfite as an agent to be applied to the nasal mucosa in order to decrease the pH and thereby inhibit virus. It is stated that this intervention “can serve to abort the development of these initial symptoms into a full blown rhinovirus common cold infection”.

U.S. Pat. No. 6,641,801 describes a gargle method using an ethanol based mouthwash as an antiviral agent to “block the common cold”

USPTO application # 20070110676 by Clymer, et al. at Proctor and Gamble describes a guaiacol and mucoadhesive agent combination of acid pH used to create an environment hostile to respiratory viruses which allows it to be effective in preventing the onset of symptoms of the common cold or influenza.

Recently, Proctor and Gamble in Great Britain has marketed a nasal spray based on this patent which purports to alter the course of the common cold. This gel is said to work by “entrapping the virus in order to inhibit attachment to respiratory mucosa”, to “inhibit the virus by its acidic pH”, and “to trigger the body to create mucus-helping to flush out the trapped, disarmed virus”. The product, which is licensed as a medical device, does not contain an alpha adrenergic agent, as Oxymetazoline, nor has been recommended for use with such an agent. It is marketed as “Vicks First Defence (sic)” and has no drug ingredients.

Of interest and of some relevance to this patent request, Proctor and Gamble has marketed two different products in the United States as “Vicks Early Defense”. One of these is a nasal spray which is recommended for use “at the first sign of a stuffy nose” or cold to “stop nasal congestion before it stops you”. This product contains oxymetazoline in a microgel formulation. There is no assertion that early use of this product will achieve anything other than the relief of congestion. Furthermore, there is no recommendation for its use when cold onset begins with any of the other symptoms that often precede congestion, and there is no statement that it can interrupt the development or change the course of the cold.

Proctor and Gamble also markets a non-alcohol based foaming gel as a hand sanitizer under the name “Vicks Early Defense”. This is intended to decrease microbial transmission which might occur from hand contact with contaminated surfaces and does not relate, other than in name, to either their British gel product or their oxymetazoline spray.

As noted previously, any intervention that truly alters the course of infection or the occurrence of the common cold would have dramatic societal impact. The individual suffering from the infection would benefit, but benefit would also be seen due to a decrease in secondary transmission; that is, the transmission of infection to family members, co-workers, schoolmates, and inadvertent contacts.

In order to achieve broad usage and achieve the benefits described, an agent must be simple to use, easy to understand, and must be non toxic. The invention detailed in this application meets these criteria.

### BRIEF SUMMARY OF THE INVENTION

The object of this invention is to use a salt of an alpha adrenergic agent (agonist), as oxymetazoline hydrochloride, in long acting preparation in such a manner to cure/interrupt the development of the common cold syndrome in adults and older children.

Clinical observation of this therapeutic intervention reveals that use of a long acting alpha adrenergic agent within one (1) hour of the time of onset of first symptom(s) caused by the infection can abort development of the common cold syndrome and result in a cure of the infection. The initial symptom(s) that alerted the user to the cold onset routinely abates over 6-8 hours and further symptoms do not develop. When therapy is delayed until one (1) hour to three (3) hours after symptom onset, patients may experience minor cold symptoms which can persist for 36 to 48 hours.

Alpha adrenergic agents are commonly recommended by Physicians and used by the general public for the relief of nasal congestion and runny nose which are associated with the common cold. What is novel in this invention is that early use of this agent, even in the absence of nasal congestion, changes the course of the illness. If the intervention is initiated at the time of, or shortly after, the onset of first symptom(s), the common cold can be cured or significantly altered. First symptoms may include sore throat, sneezing, cough, fever, chills, nasal congestion, and tiredness or a combination of these.

An alpha adrenergic agent is not generally used at a time when the first cold symptom occurs. It is also not usual for individuals to initiate therapy with these agents before symptoms are significant or “full blown”. In our experience, intervention at this stage of the infection will not cure the infection and can only ameliorate or shorten the duration of some symptoms.

Studies of Alpha Adrenergic agents have revealed that they do exhibit antiviral properties and that they can inhibit rhinoviral growth in tissue culture. (F. Sacher et al, Deutsche Apotheker Zeitung, no 5, Feb. 3, 2005 pp 111-112). In addition, oxymetazoline has been shown to decrease the duration of rhinitis from six (6) to four (4) days when compared with saline placebo in subjects with acute rhinitis. (S. Reinecke and M. Tschaiken, Fortschr Med Originalien Nr III/2005, pp 13-118). These studies demonstrate that oxymetazoline has activity against the cold virus and has benefits in treating the common cold that have not been previously recognized. Together, they help support the claim of the present invention.

This novel invention and the effectiveness of this agent in curing the common cold have not been previously recognized because this class of agents, though used for many years to treat the symptoms of the common cold, has not been initiated early enough after the development of symptoms to allow investigators to recognize its ability to interrupt or cure this infection.

### DRAWINGS

Not Applicable

### DETAILED DESCRIPTION OF THE INVENTION

### Formulation

The invention consists of the amount of oxymetazoline hydrochloride or a pharmaceutically acceptable salt thereof which is sufficient to cause nasal decongestion. This varies from about 0.001 to about 0.2% by wt/vol. However, ranges of 0.01% to 0.1% of the total composition may be effective. A concentration of 0.05% by wt/vol. is felt optimal for use in adults and children above five years of age. It is not clear whether lower concentrations may be as effective in curing colds and may allow use in younger children or in adults who have contraindications to use at higher concentrations.

an aqueous carrier comprising:
70 to 90% by weight/volume of water;
0.10 to 5.00% by weight/volume of an aromatic alcohol;
0.01 to 0.3% by weight/volume of a non-mercurial antimicrobial preservative;
0 to 10% by weight/volume of a moisturizing agent;
a sufficient amount of a pharmaceutically acceptable buffer to maintain the pH of the composition within the range of about 4.00 to 6.00;
and comprising other agents as deemed necessary to enhance flavor or to enhance the functional capabilities of the primary agent.

The means of commercial production of a long acting alpha adrenergic nasal sprays are well known within the pharmaceutical community, and the embodiments of these may comprise multiple and various nonessential agents which are added to the formulations to enhance the acceptability and functionality of the product. These are not the subject of this invention, but this invention should be deemed as inclusive rather than exclusive of these options.

Embodiment of the present invention can also include compositions that comprise a virus inactivation agent. The inactivation agent can temporarily or permanently prevent virus infectivity to result in prevention and treatment of cold symptoms.

Embodiment of the present invention can comprise one or more inactivation agents, provided that the total concentration of inactivation agent is from about 0.01% to about 20%, preferably from about 0.05% to about 10%, more preferably from about 0.10% to about 5%, by weight of the composition. Inactivation agents suitable for use herein include metal compounds, surfactants, chelating agents, pyroglutamic acid, and mixtures thereof.

Examples of metal compounds suitable for use as an inactivation agent herein include those metal compounds commonly referred to as “metal salts” which comprise metal ion substituents selected from the group consisting of manganese (Mn), silver (Ag), zinc (Zn), tin (Sn), iron (Fe), copper (Cu), aluminum (Al), nickel (Ni), cobalt (Co), and mixtures thereof. Preferred metal compounds include those metal compounds which contain Cu, Fe, or Zn metal ions, or combinations thereof. Examples of such metal compounds include the metal compounds referred to as salicylates, fumarates, benzoates, glutarates, lactates, citrates, malonates, acetates, glycolates, thiosalicylates, adipates, succinates, gluconates, aspartates, and glycinates.

Embodiment of the present invention can include the administration of compositions that comprise a nasal secretion agent that provides for the removal of viruses and/or viral strains from the respiratory tract area, especially from the nasal cavity. The nasal secretion agent stimulates a mild rhinorrhea that may cause virus particles and inflammatory mediators to be washed away from affected cell receptors located in respiratory tract areas.

The present invention can comprise one or more nasal secretion agents, provided that the total concentration of nasal secretion agent is from about 0.001% to about 10%, preferably from about 0.005% to about 5%, more preferably from about 0.01% to about 1%, by weight of the composition. Nasal secretion agents suitable for use herein include organic acids, aromatic plant extracts, hypertonic solutions, and mixtures thereof.

While the alpha agonist used in these observations was oxymetazoline in long acting formulation, other alpha agonists as phenylephrine, pseudoephedrine, phenylpropanolamine, napthazoline, tramazoline, and xylometazoline in both long and short acting forms and as water based and gel formulations are exemplary compounds of this invention.

While oxymetazoline was used as a single agent in these observations, it should be recognized that the combination use of this agent in a manner as described in this invention with other agents may provide either additive or synergistic effect. While description has been made of an intervention using a single agent, those skilled in the art will recognize that combination of this agent with other compounds when used in the manner described will remain within the spirit and claim of this invention.

These agents may include but are not limited to antihistamines, anti-inflammatory agents, antiviral agents, metallic compounds and acids and inhibitory gels. Combination of this medication with a microgel formulation might provide an additive or synergistic effect and is a extension of this invention and is, therefore, provided as a treatment to abort/arrest/cure the common cold

### Administration

The agent is administered in water base with one (1) to three (3) sprays applied to each nostril and should be “sniffed” as deeply as possible into the nose and nasopharynx. If sore throat is the initial symptom which prompts use of the agent or one of the initial symptoms, sniffing the agent more deeply into the pharynx appears to provide optimal effect. In patients whose illness begins with a sore throat a water based spray may be the preferred treatment. A direct application of the agent by spray into the posterior pharynx is an acceptable treatment option.

A standard nasal spray dispenser consisting of a reservoir for liquid which is compressible by fingertip pressure is the dispenser of choice. It should contain a tapered nozzle which can be placed into the nostril. The therapeutic agent is dispensed into the nasal cavity by compression of the reservoir. Forceful inhalation is recommended after instillation in order to facilitate uniform and broad distribution of the agent.

An acceptable option would allow a liquid based formulation to be applied by dropper, by propellant based aerosol, by perfume bottle dispenser or by other vehicles know and unknown that would allow adequate distribution of the agent to the mucosal surfaces.

Having described my invention, what I claim as new and desire to secure through application for patent is as follows:

1. A method of treating the common cold in such a fashion as to abort/arrest development of the common cold syndrome. As such, the treatment purpose is to cure the common cold. This method comprises the following steps:
providing a nasal spray containing oxymetazoline to an individual in any of various therapeutic concentrations and in any of various formulations via the anterior nares and/or mouth so that the agent makes contact with the mucosa of the nasopharynx and/or pharynx.
providing the stated agent prior to the development of the cold syndrome, ideally within one hour of the onset of first cold symptom(s).
providing this agent, commonly used for its decongestant effect, with the intent of changing the course of the common cold and blocking the development of the common cold syndrome. The intent of this intervention is to seek cure of the syndrome rather than amelioration of symptoms.
providing this agent with a timing that is not commonly recognized as important with the intent of achieving an effect different than that commonly thought achievable.
